(19) United States
(12) Patent Application Publication
(10) Pub. No.: US 2006/0133996 A1
(43) Pub. Date: Jun. 22, 2006

(54) Method and apparatus for determining effectiveness of sunscreens and other skin preparations in shielding human skin from uva radiation

(76) Inventors: Rachel Mary Haywood, Pinner (GB); Peter Wardman, Amersham (GB); Roy Sanders, London (GB); Claire Linge, London (GB)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 10/533,617

(22) PCT Filed: Oct. 28, 2003
(86) PCT No.: PCT/GB03/04637

§ 371 Date: Nov. 4, 2005

(30) Foreign Application Priority Data
Oct. 31, 2002 (GB) 02254084

(51) Int. Cl. A61K 49/00

(52) U.S. Cl. 424/9.1; 424/59

(57) ABSTRACT

The present invention provides a method and apparatus for measuring the effectiveness of a sunscreen composition or other skin preparation in reducing the exposure of human skin to UVA radiation, wherein differential electron spin resonance spectroscopy is used to quantify the extent of UVA-induced ascorbate or other measurable radical production in the shielded skin, in comparison with reference, preferably unshielded, skin.

### FIELD OF THE INVENTION

The present invention relates to a method and an apparatus for determining effectiveness of sunscreens and other skin preparations in shielding human skin from solar and artificial UVA radiation. The present invention also relates to the use of data collected from such determinations.

### BACKGROUND OF THE INVENTION

It is clearly established that ultra-violet (UV) wavelengths of sunlight and UV lamps (below about 400 nm) cause premature skin ageing and are carcinogenic, contributing towards the formation of skin malignancy in the form of squamous and basal cell carcinoma, and malignant melanoma. Three primary regions of ultra-violet radiation have been identified and classified according to their effects on human skin, namely WVA (wavelength approximately 320-400 nm), UVB (wavelength approximately 290-320 nm) and UVC (wavelength approximately 230-290 nm). Basal and squamous cell carcinomas are predominantly a result of direct damage to the DNA by interaction with UVB photons [Linge C., Relevance of in vitro melanocytic cell studies to the understanding of melanoma, Cancer Surveys, 26, 71-87 (1996)]. By contrast, malignant melanoma is now being linked with UVA [Setlow, R. B., Grist, E., Thompson, K. and Woodhead, A. D, Wavelengths effective in induction of malignant melanoma, Proc. Natl. Acad. Sci. USA 90, 6666-6670 (1993); Moan, J., Dahlback, A., and Setlow, R. B., Epidemiological support for a hypothesis for melanoma induction indicating a role for UVA radiation, Photochem. Photobiol. 70, 243-247 (1999); Wang, S. Q., et al. Ultra-violet A and melanoma: a review, J. Am. Acad. Dermatol., 44, 837-846 (2001)], and UVA induces the production of free radicals [Scharfettner-Kochanek K., Wlaschek, M., Brenneisen, P., Schauen, M., Blaudschun, R., and Wenk, J., UV-induced reactive oxygen species in photocarcinogenesis and photoageing, Biol Chem., 378, 1247-1257 (1997); Packer, L., Ultra-violet radiation (UVA, UVB) and skin antioxidants in “Free Radical Damage and its control”, Rice-Evans, C. A. and Burdon, R. H. (Eds.) 1994 Elsevier Science B. V.], which can indirectly damage DNA. UVC radiation is effectively absorbed by the atmosphere, and is present only in small amounts in sunlight reaching the earth.

It is common to apply a sunscreen to the skin in order to shield the skin from UV radiation. Certain cosmetics also claim to offer some UV protection to the skin. Generally speaking, sunscreens and UV-protective cosmetics comprise a carrier, normally in the form of a liquid, cream, wax, paste, gel or the like, and an active UV absorbing or reflecting agent dissolved, mixed or suspended therein. The UV absorbing or reflecting agent can be an organic or inorganic chemical with the capacity to absorb or reflect incident radiation in the UV wavelength range. The sunscreen is applied by the human user to his/her skin, typically by spreading to form a thin covering layer on the skin.

Even when properly applied, sunscreens are not capable of completely blocking all UV radiation from reaching the human user's skin. To provide guidance to users as to the efficacy of a sunscreen against UVB radiation, the so-called “sun protection factor” (SPF) rating system is used. The SPF is a multiplication factor, representing the degree of lengthening of the time period before the onset of erythema obtained by using the sunscreen at the recommended application level. Thus, for example, a person whose skin type is such that erythema would arise after 15 minutes at a given UV intensity without skin protection would, after application of a sunscreen having a SPF of Z, be able to tolerate exposure for 15×Z minutes before the onset of erythema.

Erythema is a downstream inflammatory response to primarily UVB radiation, so that the SPF rating of a sunscreen is predominantly an indicator of the sunscreen's efficacy at screening UVB radiation. In practice, however, users cannot accurately know what their unprotected erythema onset time would be on any particular day, so accurate application and reapplication of sunscreens is practically impossible. In any event, SPF is not a reliable indicator of the protection provided by sunscreens against carcinogenesis and e other long-term adverse effects, particularly those induced by “indirect” damage from UVA exposure, and which are not related to erythema onset.

Despite the extensive use of sunscreens during the last two decades, the incidence of skin cancers is still increasing, and sunscreen use has been directly correlated with increased skin cancer risk [Autier, P., et al., Melanoma and the use of sunscreens: an EORTC case-control study in Germany, Belgium and France, Int. J. Cancer, 61:749-755 (1995); Vainio, H. and Bianchini, F., Cancer-preventative effects of sunscreens are uncertain, Scand. J. Work Environ Health, 26, 529-531 (2000); Azizi, E. et al. Use of sunscreen is linked with elevated naevi counts in Israeli school children and adolescent,. Melanoma Res., 10, 491-498 (2000)]. The causes appear to include: inadequate application [Wuilf, H. C., Stender, I. M., and Lock Andersen, J., Sunscreens used at the beach do not protect against erythema: a new definition of SPF is proposed, Photodermatol. Photoimmunol. Photomed, 13, 129-132 (1997); Gaughan, M. D. and Padilla, R. S., Use of a topical fluorescent dye to evaluate effectiveness of sunscreen application, Arch. Dermatol., 134, 515-517 (1998); Stokes, R. and Diffey, B., How well are sunscreen users protected? Photodermatol. Photoimmunol. Photomed., 13, 186-188 (1997)]; lack of durability of the application; the lack of, or inadequacy of UVA filters in sunscreen preparations combined with prolonged sunbathing [Autier, P., et al. (1995), see above]; the photoinstability of sunscreen filters that results in reduced protection; and the production of reactive free-radicals or mutagens within the cream [Flindt-Hansen, H., Nielsen, C. J., and Thune, P., Measurements of the photodegradation of PABA and some PABA derivatives, Photodermatol., 5, 257-261 (1988); Gasparro, F. P., The molecular basis of LTV-induced mutagenicity of suncreens, FEBS, 336(1), 184-185 (1993); Shaw, A. A., Wainschel, L. A and Shetlar, M. D., Photoaddition of p-aminobenzoic acid to thymine and thymidine, Photochem. Photobiol., 55, 657-663 (1992); Knowland, J., McKenzie, E. A., McHugh, P. J., and Cridland, N. A., Sunlight-induced mutagenicity of a common sunscreen ingredient, FEBS, 324(3), 309-313 (1993); Dunford, R., Salinaro, A., Cai, L., Serpone, N., Horikoshi, S., Hidaka, H., Knowland, J. Chemical oxidation and DNA damage catalysed by inorganic sunscreen ingredients, FEBS Letters 418, 87-90 (1997)]. It seems likely that the SPF rating system, in the absence of a comparable guidance system for UVA radiation, may actually be encouraging excessive exposure to UVA radiation, particularly by fair-skinned northern Europeans without adequate skin protection either from evolution or available sunscreens.

A range of methods have been attempted, in order to measure UVA radiation, particularly with the aim of introducing an easily understood rating system for sunscreen and other skin preparations, whereby users could effectively and with reasonable accuracy control their daily exposure to UVA radiation [Cole, C., Sunscreen protection in the ultraviolet A region: how to measure the effectiveness, Photodermatol. Photoimmunol. Photomed. 17, 2-10 (2001)]. Some measurement methods based on erythema or pigmentation responses have been used commercially, but there remains a need for a more quantitative assay method.

U.S. Pat. No. 6,348,694 (Gershteyn et al) describes a method and apparatus for determining the ability of skin to withstand exposure to harmful radiation, and a safe exposure time of the skin. This method and apparatus are said to monitor in real time the skin darkening response to the incident radiation, and the intensity of the radiation reaching the skin (allowing for the effect of any sunscreen), from which data a safe exposure time is calculated. While such a system can help a user who is unaware of the intensity of radiation on a particular day, and unaware of his/her natural (unprotected) tolerance time before onset of erythema, the system does not satisfactorily measure damaging effects of incident UVA radiation.

The disclosures of the above prior publications are incorporated herein to the extent permitted by applicable law.

It is known that UVA radiation induces in human and animal skin an oxidative stress which results in the formation of free radicals, although the responses are different between human and animal skin and analogies between the two must be treated with caution. The free radicals formed upon UV-irradiation of skin (and which are associated with DNA and protein damage) are not usually directly detectable at room temperature. An exception to this, however, is the ascorbate radical, which is formed when ascorbate (vitamin C—a cellular antioxidant) reacts with free radicals. In an article entitled “Ascorbate Radical: A Valuable Marker of Oxidative Stress” published in “Analysis of Free Radicals in Biological Systems”, 1995, pp 145-163, the disclosure of which is incorporated herein by reference to the extent permitted by applicable law, Buettner and Jurkiewicz describe how the spin resonance spectroscopic technique of electron paramagnetic resonance (EPR) can be used to measure the level of ascorbate radical induced in, amongst other things, mouse skin exposed at room temperature to UV radiation at wavelengths above 300 nm (UVA and UVB radiation) (pages 155-156). It is not stated how, if at all, the EPR signal amplitude correlates with the level of UVA exposure.

In a subsequent article entitled “EPR Detection of Free Radicals in UV-Irradiated Skin: Mouse versus Human” (Photochemistry and Photobiology, 1996, 64(6), pp 918-922), the disclosure of which is incorporated herein by reference to the extent permitted by applicable law, the same authors report a number of differences observed between mouse and human skin in the free radical production response to UV radiation.

As far as human skin results are concerned, it is reported (page 919, column 2) that exposure of human skin, which had been frozen at 77K (−196° C.) between excision and use, to predominantly UVA radiation (UVA fluence rate 3.5 mW/cm² as compared with UVB fluence rate 14 μW/cm²) caused an approximately fourfold increase in the ascorbate radical signal height almost immediately. This signal height was maintained with some fluctuation at generally the same, or slightly increasing, level while the UV radiation was maintained (FIG. 2). A lesser degree of increase is reported for mouse skin (page 919, column 2).

The present invention is based on our surprising finding that the UVA-induced production of ascorbate radical in a sample of human skin or the like returns rapidly to the background level in the skin on removal of the UVA source, and is then re-established in a quantitatively comparable manner on subsequent re-exposure to the UVA radiation after a time period of the order of minutes.

This finding enables a differential electron spin resonance spectroscopic UVA-induced ascorbate radical assay to be performed, the assay including a first measurement taken on an unshielded sample of human skin or the like and a second on the same or a comparable sample shielded with a measured dose of sunscreen or other skin composition to be tested, whereby a direct quantitative measure of the effect of the sunscreen or composition on UVA-induced free radical production in the skin can be obtained.

From such a quantitative result, a UVA sun protection factor (UVASPF) can be assigned to sunscreens, proportional to the extent of reduction in the UVA-induced free radical production observed following application of the sunscreen. Because the rate of generation of free radicals in the skin is found to be substantially constant for a given intensity of incident UVA radiation, a UVASPF of, for example, 5, would indicate that using the sunscreen at the recommended application level for a certain time period (t) would result in a level of UVA-induced oxidative stress in the skin equivalent to that resulting from a time period t/5 of unshielded exposure to the same intensity of UVA radiation.

Again surprisingly, and concerningly, we find that the SPF values stated on commercially available sunscreens do not equate with UVASPF values assigned using the method of the present invention, the UVASPF values as measured by the method according to the present invention being substantially lower than the publicly stated SPF values, suggesting that many people, sunbathing with UVB protection for prolonged periods pre-erythema, are being exposed to substantially more UVA radiation per day than their skin is adapted to tolerate. The method of the present invention, coupled with international agreement as to a safe total daily exposure to UVA, would enable a universal UVASPF system to be introduced, whereby a minimum UVASPF value and a maximum daily period in the sun could be recommended for sunscreens or other skin preparations, dependent on the particular latitude, season and climate.

### BRIEF DESCRIPTION OF THE INVENTION

According to a first aspect of the present invention, there is provided a method for measuring the effectiveness of a sunscreen composition or other skin preparation in reducing the exposure of human skin to UVA radiation, the method comprising:

irradiating a sample of human skin or of an effective substitute therefor (herein: “skin”), shielded with the sunscreen composition or other skin preparation to be tested, with UV radiation comprising UVA wavelengths, and determining by electron spin resonance (ESR) spectroscopy the level of induced production of ascorbate radical in the shielded skin; and

determining a quantitative measure of the effectiveness of the sunscreen composition in reducing the exposure of human skin to UVA radiation by comparison of the said level of ascorbate radical production in the shielded skin with the level of ascorbate radical production induced in reference skin under substantially quantitatively comparable conditions.

The test and reference samples of skin may be the same or different, as described in more detail below. Where the samples are different, they should be as closely comparable as possible in terms of their ascorbate radical response under exposure to UV radiation. Most preferably, the reference skin is the same skin as the test sample, used under essentially the same ESR conditions as the test, but without a sunscreen shield or with a standard shield the relevant characteristics of which are known, provided that the necessary quantitative comparison can be made to determine the UVA screening effectiveness of the sunscreen under test.

According to a second aspect of the present invention, there is provided an apparatus for testing the effectiveness of a sunscreen composition or other skin preparation in reducing the exposure of human skin to UVA radiation, the apparatus comprising:

at least one sample of human skin or of an effective substitute therefor (herein: “skin”);

a source of UV radiation comprising UVA wavelengths;

means for determining by electron spin resonance (ESR) spectroscopy the level of induced production of ascorbate radical in a skin sample on exposure of the skin to the UV radiation;

means for shielding a skin sample with the sunscreen composition or other skin preparation to be tested; and

means for determining a quantitative measure of the effectiveness of the sunscreen composition or other skin preparation in reducing the exposure of human skin to UVA radiation by comparison of the level of ascorbate radical production in the shielded skin with the level of ascorbate radical production induced in reference skin under substantially quantitatively comparable conditions.

The means for shielding a skin sample with the sunscreen composition may be adapted for use with the same skin sample as used for the determination of ascorbate production in unshielded skin, or with a different sample from that used for the determination of ascorbate production in unshielded skin, as described in more detail below. Where the shielded and unshielded samples are different, they should be as closely comparable as possible in terms of their ascorbate radical response under exposure to UV radiation.

According to a third aspect of the present invention, there is provided a method for assigning a UVA sun protection factor (UVASPF) or a free radical protection factor (FRPF) to a sunscreen composition or other skin preparation, which method comprises measuring the effectiveness of the sunscreen composition or other skin preparation in reducing the exposure of human skin to UVA radiation, using the method of the first aspect of the present invention or the apparatus of the second aspect of the present invention, expressing the said effectiveness as the fraction (f) of unshielded UVA-induced ascorbate radical production exhibited by the shielded skin, and assigning the UVASPF or the FRPF to the composition or preparation by virtue of the relationship:
UVASPF (or FRPF)=1/f.

According to a fourth aspect of the present invention, there is provided a sunscreen composition or other skin preparation, to which a UVASPF or FRPF has been assigned according to the third aspect of the invention.

According to a fifth aspect of the present invention, there is provided a sunscreen composition or other skin preparation, preferably for application to the skin at least once per day, to which a UVASPF or FRPF has been assigned according to the third aspect of the invention, which UVASPF or FRPF is above the safe minimum UVASPF or FRPF for the latitude, season and/or climate in which the composition or preparation is to be used, calculated having regard to a safe maximum daily exposure to UVA radiation and an assumed, expected or likely actual daily exposure to UVA radiation at that latitude, season and/or climate.

In place of the ascorbate radical, other radicals produced in skin in response to incident UVA wavelengths may be measured according to the present invention. Where such radicals are too short-lived to be stable over the period required for the measurements, a spin trap molecule will be used to stabilise the radical sufficiently for the measurements. For effective use in the present invention, the resultant adduct of the spin trap molecule with the short-lived radical should preferably have a substantially quantitatively stable lifetime of at least about 100 s, preferably at least about 1000 s. The present invention embraces modifications of the first to fifth aspects described above, in which measurement of the ascorbate radical is replaced by, or conducted alongside, measurement of one or more other radicals produced in skin in response to incident UVA wavelengths, or adducts thereof with spin trap molecules, provided that the said other radical or adduct is substantially quantitatively stable over a lifetime of at least about 100 s and is measureable using ESR spectroscopy. The term “measurable radical” used herein refers to ascorbate and all other radicals produced in skin in response to incident UVA wavelengths, which are either substantially quantitatively stable over a lifetime of at least about 100 s and are measureable using ESR spectroscopy or can form an adduct with a spin trap molecule, the adduct being substantially quantitatively stable over a lifetime of at least about 100 s and being measureable using ESR spectroscopy.

According to a sixth aspect of the present invention, there is provided the use of differential ESR spectroscopy in a method for measuring the effectiveness of a sunscreen composition or other skin preparation in reducing the exposure of human skin to UVA radiation. The differential ESR spectroscopy is applied to measurable radicals in accordance with the invention, and is preferably, but not exclusively, used to quantify UVA-induced ascorbate radical production in skin shielded by the said composition or other skin preparation, in comparison with unshielded skin.

### DETAILED DESCRIPTION OF THE INVENTION

The expression “human skin or an effective substitute therefor” used herein refers to human skin tissue or discrete human skin cells, and the tissue or discrete cells of any animal skin or other biological material which provides a quantitative measurable differential measurable (e.g. ascorbate) radical response under UVA radiation and is therefore equivalent to human skin for the purposes of this invention. Suitable animal skin may, for example, include natural animal skin and animal skin comprising genetically modified (e.g. humanized) cells. The skin may, for example, comprise chemically modified or cultured skin cells.

The expression “sunscreen composition or other skin preparation” used herein refers to any composition adapted or intended to have an effect of reducing the intensity of solar or artificial UV radiation incident on human skin when applied, usually directly, to that skin. Such compositions may include sunblocks, suncreams, sun lotions, anti-ageing creams, anti-wrinkle creams, moisturising creams, and general UV-protective cosmetic and medicinal creams or lotions. Generally speaking, such materials comprise a carrier, normally in the form of a liquid, cream, wax, paste, gel or the like, and an active UV absorbing or reflecting agent dissolved, mixed or suspended therein. The UV absorbing or reflecting agent can be an organic or inorganic chemical with the capacity to absorb or reflect incident radiation in the UV wavelength range. Such materials and components are well known in the art, and a detailed description is not required here.

The expression “UV radiation” used herein refers to electromagnetic radiation having a wavelength in the range between violet light and long X-rays i.e. about 4-450 nm, for example about 4-400 nm.

The expression “UVA radiation” used herein refers to UV radiation in the wavelength range 320-450 nm, for example 320-400 nm. Within this range, wavelengths in the range 320-360 nm may be termed UVA II, and wavelengths within the range 360-450 nm may be termed UVA I.

The expression “UVB radiation” used herein refers to UV radiation in the wavelength range 290-320 nm.

The expression “Electron Spin Resonance” or “ESR” used herein refers to spectroscopy in which a resonance response is measured on exposure of unpaired electrons in a tested material to radiation of measurable frequency and wavelength. ESR spectroscopy is sometimes referred to as electron paramagnetic resonance or EPR spectroscopy.

The expression “effectively the same ESR conditions” used herein refers to testing and measurement conditions which are, within the acceptable limits of accuracy, capable of providing quantitatively comparable results as between two different assays.

The expression “ascorbate radical” used herein refers to the radical commonly denoted as (Asc.)-, which has the structure shown below:

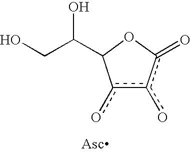

The Skin

The skin used in the present process is preferably freshly (i.e. less than 24 hours previously) excised human skin tissue, which is maintained at a temperature above 0° C. and most preferably between about 0 and about 6° C. between excision and use. Less preferably, the skin may be stored between excision and use, e.g. at a temperature below about 0° C. The use of fresh skin avoids the build-up of background levels of free radicals and is found to produce an acceptably constant assay reading over the length of time taken to collect the data, compared with the methodology used by Buettner and Jurkiewicz in their 1996 paper and reported there in FIG. 2.

It is preferred to minimise variability in the skin samples used. The present invention therefore preferably uses similar tissue from a standard part of the body, most preferably a part where the skin has had relatively low past exposure to UV radiation e.g. Caucasian female breast skin tissue. Alternatively, standardised cultured, cloned or otherwise engineered skin may be used, selected to have a high degree of reproducibility from sample to sample.

The UV Source

The method of the present invention is found to enable a quantitative assay of the UVA-protective effects of a sunscreen or other skin preparation, at sub-erythemal exposure doses of UVA radiation, which is comparable with the intensity of natural sunlight. Such UVA exposure levels are substantially lower than in many of the previously available assays (see the Cole paper, referred to above). Typical UVA exposure doses in the method of the present invention will be equivalent to sunlight of between about 0.5 to about 0.9 Minimum Erythema Doses (MED) for Caucasian skin. It is preferred that this dose of UVA radiation is delivered over a period of time in the order of tens to hundreds of seconds, typically about 100 s, preferably at a UVA radiation intensity in the normal solar range, e.g. in the general range about 1 to about 100 mW/cm². The UV radiation incident on the sample preferably comprises predominantly UVA wavelengths, e.g. UVA wavelengths at a fluence rate more than 100, more preferably more than 200, times the UVB fluence rate. The UVA radiation incident on the sample preferably has an intensity comparable that of sunlight i.e. approximately 1.3 mW/cm².

The source of UV radiation comprising UVA wavelengths preferably consists of a UV lamp or solar simulator which, according to the manufacturer's specification, emits UVA radiation at the desired intensity and wavelengths. Suitable filters may be used to remove unwanted wavelengths, in conventional manner. An example of a suitable low intensity UVA lamp is the super high pressure 100 W Nikon mercury lamp, model LH-M1100CB-1.

The Ascorbate Assay and the Assay Apparatus

The means for determining by electron spin resonance (ESR) spectroscopy the level of induced production of ascorbate radical in the skin on exposure of the skin to the UV radiation preferably consists of an ESR instrument, including sample container and sample handling devices, and associated signal processors and peripherals. Such an instrument, processors and peripherals are commercially available and the principles and materials of their construction and operation are known. An example of a suitable ESR instrument is the Bruker EMX spectrometer, available from (available from Bruker BioSpin GmbH, Division IX, Silbersteifen D-76287 Rheinstetten/Karlsruhe, Germany, Tel: ++49-721-5161-141, www.bruker.de. An example of a suitable sample container is the tissue cell WG 806-B-Q, available from Wilmad Lab Glass (1002 Harding Hwy., POB 688, Buena N.J. 08310 USA, Tel: ++856 697 3000, www.wilmad.com). It is preferred that the apparatus and method of the present invention are operated at approximately room temperature, i.e. in the temperature range of about 10 to about 30° C.

The level of induced production of ascorbate radical in the skin may suitably be quantified from the height of the characteristic low-field ascorbate radical doublet peak (at a magnetic field between 3452 and 3456 Gauss), the peak height being determined from a base reference level corresponding to the midpoint signal trough between the two peaks of the doublet 3453.5 G.

The means for shielding a sample of skin with the sunscreen composition or other skin preparation to be tested preferably comprises a UV-inert support member having a surface capable of receiving and retaining a measured coating weight of the sunscreen composition or other skin preparation to be tested. By “UV-inert” is meant a support member which does not affect the quantitative nature of the assay. The support member is preferably transparent to UV, or at least to UVA, radiation, and preferably does not itself generate free radicals on exposure to UV radiation. The support member may suitably consist of a quartz slide locatable in the path of the UV radiation between the UV source and the skin sample, more preferably a cover slide adapted for use with the container (e.g. sample cell) for the skin sample. The sunscreen or other skin preparation used in the present invention is preferably applied approximately in accordance with the manufacturer's recommended dosage. The manufacturer's recommended conditions of application—e.g. a drying period—are also preferably observed.

The means for determining a quantitative measure of the effectiveness of the sunscreen composition or other skin preparation in reducing the exposure of human skin to UVA radiation, by comparison of the levels of ascorbate radical production in the shielded and reference skin samples, preferably comprises electronic signal processors and conventional associated electronic apparatus adapted to measure the differential signal height between the samples and to display the result as a readout and/or printout in generally conventional manner. The provision of such apparatus and associated controlling software will be well within the capacity of one of ordinary skill in this art, and does not require further elaboration.

The apparatus according to the present invention may suitably be provided with the skin sample pre-installed, or may be adapted so that replacement or alternative skin samples can be easily substituted for an existing installed skin sample, without any need for handling of the sample. For example, one or more skin sample may be provided to a user of the apparatus in the form of a sealed “cassette” consisting of an ESR cell or other container holding the skin sample on a suitable mounting within the cell or container. The support member holding the sunscreen composition or other skin preparation to be tested refill suitably be located between the cassette and the UV source. Where it is desired to test sunscreen compositions on a range of different skin types (e.g. Caucasian, Afro-Caribbean, Chinese, etc.), an appropriate one of a series of interchangeable cassettes can simply be inserted into the apparatus. The apparatus according to the present invention may, for example, be used in a laboratory or a sunscreen manufacturing facility for quality control purposes.

Other Measurable Radicals

As described generally above, and as set out in more detail in the 1996 Photochemistry and Photobiology article by Jurkiewicz & Buettner referred to above, the radical measured by the ESR technique according to the present invention may be selected from one or more other measurable radicals, and the measurement thereof may be conducted in place of, or alongside, the measurement of the ascorbate radical.

Generally speaking, most or all other radicals quantitatively produced in human skin in response to incident UVA wavelengths are too short-lived to be measurable as such in the context of a method or apparatus for testing the effectiveness of a sunscreen composition or other skin preparation in reducing the exposure of human skin to UVA radiation. Therefore, in those cases a spin trap molecule must be contacted with the skin, so that an adduct of the radical with the short-lived radical is formed in the skin, which adduct is substantially quantitatively stable over a lifetime of at least about 100 s, preferably at least about 1000 s, and is measureable using ESR spectroscopy.

Suitable spin trap molecules for use in the present invention include, for example, 5,5-dimethylpyrolline-N-oxide (DMPO), 3,5-dibromo-4-nitro-benzenesulplhonic acid (DBNBS), N-t.butyl-α-phenylnitrone (PBN) or α-(4-pyridyl-1-oxide)-N-t.butyl-phenylnitrone (POBN), which are effective to stabilise radicals produced in the skin on UV exposure, e.g. oxygen radicals such as superoxide, alkoxyl, SO₃.⁻ and hydroxyl, and carbon-centred radicals derived from proteins and lipids such as alkyl radicals.

Further details of the techniques for quantitatively measuring spin trap adducts of UVA-generated skin radicals using ESR spectroscopy may be found in, for example, the Buettner and Jurkiewicz articles referred to above and incorporated herein by reference. The selection of suitable spin trap molecules and the techniques for handling, using and measuring them, will in any event be well within the capacity of one of ordinary skill in this art, and do not require detailed elaboration here.

The present invention will now be described in more detail, but without limitation, with reference to the specific Examples described below and illustrated in the accompanying drawings.

### BRIEF DESCRIPTION OF THE DRAWINGS

FIG. 1 illustrates (a) an ESR characteristic (low-field absorbance) ascorbate radical spectrum of human Caucasian skin immediately upon UV irradiation without shielding; (b) the ESR spectrum of sunscreen alone at 2 mg/cm², application amount, to show that radicals formed in the sunscreen, at this application, do not interfere with the ascorbate radical signal; and (c) an ESR time scan of the signal intensity of the low-field absorbance peak of the ascorbate radical in response to irradiation, monitored with time.

FIG. 2 illustrates typical ESR spectra of UV-irradiated human Caucasian skin (each horizontal row is a different skin sample), both unshielded (left-hand column) and shielded (right-hand column) with a SPF factor 30, four star (following the star system for rating UVA protection currently used in the United Kingdom), suncream, different sunscreen application levels.

FIG. 3 illustrates (a) a plot of the percentage reduction in the characteristic ascorbate radical signal intensity against sunscreen application weight (mg/cm²) for UV-irradiated skin covered with high factor sunscreens [three brands, namely: circles—Brand 1, the SPF factor 30, four star, sunscreen used in obtaining the data shown in FIG. 2; squares—Brand 2, a commercially available SPF factor 25, four star, sunscreen; and triangles—Brand 3, a SPF 20+sunscreen not showing a star rating under the star system for rating UVA protection currently used in the United Kingdom, but nevertheless claiming UVA protection and anti-cell ageing effects]; and (b) a rank plot of percentage reduction in ascorbate radical signal intensity (n=3 for each brand at each level of application) for UV-irradiated skin covered with the sunscreens (open, hashed and shaded bars Brands 1, 2, and 3 respectively) at different levels of application. Error bars represent standard deviations.

### DETAILED DESCRIPTION OF THE DRAWINGS

Materials

Caucasian skin was obtained from consenting patients undergoing breast reduction surgery. Skin was stored in N-saline-soaked gauze at 4° C. and used within 24 hours. Prior to electron spin resonance spectroscopic analysis, skin was trimmed to remove subcutaneous fat and cut to approximately 1 cm². Sample sizes could not exceed this area due to technical limitations associated with tuning the spectrometer.

It was found that samples of skin taken from similar locations on the breast provided comparable ESR data within the accuracy of the technique. From this observation, we conclude that it is not necessary for the shielded and unshielded ESR data to be obtained on a single sample of skin, but different samples of skin can be used for the different tests. This enables the procedure to be speeded up significantly for commercial use, and the apparatus and instrumentation simplified for commercial use. It also enables relatively large batch assays of different sunscreens, or different application concentrations of sunscreens, to be run simultaneously against a single unshielded comparator sample.

Three popular brands of sunscreens, which claimed UVA protection, were randomly chosen for evaluation: brands 1, 2 and 3 used were factor 30, 25 and “20+” respectively. Two of the brands chosen had a four star UVA rating under the star system currently used in the United Kingdom, and the third was not obtained in the UK and did not have this rating (although claimed UVA protection and anti cell-ageing effects). All sunscreens were evaluated within 3 months of purchase.

Equipment

Electron-spin-resonance experiments were carried out using a Bruker Ed spectrometer (available from Bruker BioSpin GmbH, Division IX, Silbersteifen D-76287 Rheinstetten/Karlsruhe, Germany, Tel: ++49-721-5161-141, www.brulker.de) equipped with an ER 4103TM cavity and a Wilmad Glass Co. tissue cell (WG 806-B-Q) (available from Wilmad Lab Glass, 1002 Harding Hwy., POB 688, Buena N.J. 08310 USA, Tel: ++856 697 3000, www.wilmad.com). Typical ESR settings were 40 mW microwave power, 0.075 mT modulation amplitude, 2×10⁵ receiver gain, sweep time 20 s with repeated scanning (5 scans) unless otherwise indicated. LTV irradiation was carried out in situ in the spectrometer (with the cavity completely shielded by black plastic sheeting) using a super high-pressure 100 W Nikon mercury lamp (model LH-M1100CB-1) focussed on the cavity transmission window. A 5 cm water filter was used to remove infra-red radiation, together with two optical glass filters (total thickness≈1 cm) (Barr and Stroud) filtering wavelengths below 300 nm and having a 1% transmittance of UVB radiation at 300 nm and 19% at 320 nm. The UV fluence incident upon the sample within the spectrometer has been measured previously to be 3 mW (1.3 mW/cm²). The UV fluence is within levels of solar irradiation (which is 90% UVA) measured 11.00 am-3.00 pm with the same UV-actinometer (irradiated with natural sunlight through an aperture cut in black card to the same dimensions as the ESR cavity window) June-September, London, UK (direct sunlight). The UV intensity of the lamp used in these experiments is lower than levels of UVA which have been employed for solar-simulated irradiation (reported levels of solar-simulated UVA are 35 and 60-80 mW/cm²) [Ley, R, D., and Fourtanier, A., Sunscreen protection against Ultraviolet Radiation-induced Pyrimidine Dimers in Mouse Epidermal DNA, Photochem. Photobiol. (1997), 65, (6), 1007-1011; Burren, R, Scaletta, C., Frenk, E., Panizzon, R. G., and Applegate, L. A., Sunlight and carcinogenesis: expression of p53 and pyrimidine dimers in human skin following UVA I, +II and solar simulating radiations, Int. J. Cancer (1998), 76, 201-206].

Fluence measurements were taken using a potassium ferrioxalate actinometer which is a modified version of the actinometer described in Valenzeno, D. P., Pottier, R. H., Mathis, P., Douglas, R. H., Eds., Photobiological Techniques NATO ASI Series: Series A. Life Sciences 216, 50-54. The modifications are as follows: 0.006 M stock actinometer solution (0.25 ml) was irradiated directly in the flat cell held in the cavity of the ESR spectrometer, and not diluted prior to irradiation as described in step 1. In step 2, the irradiated actinometer solution was washed out of the flat cell into a 5 ml flask and made up to the mark with distilled water. The remaining procedure was as described in the Valenzeno et al reference. The fluence incident upon the sample (mol quanta per second) was calculated using equation (7-6), page 781 in “Photochemistry”, Calvert and Pitts, John Wiley and Sons, Inc. New York. This was converted to Joules per second by multiplying by the Avogadro constant, Planck's constant and the radiation frequency at 350 nm (mean frequency 300-400 nm over which the actinometer absorbs radiation). The molar absorptivity of potassium ferrioxalate (1.1×10³ Mol⁻¹ dm³ cm) is required for the calculation.

Quantitative Measurement of Free Radical Formation

The ESR technique was used to detect the ascorbate radical directly in human skin on UV-irradiation, substantially as previously published in the context of non-differential determinations [Jurkiewicz, B. A., and G. R. Buettner (1996) EPR detection of free-radicals in UV-irradiated skin: mouse versus human. Photochem. Photobiol. 64, 918-922]. The skin specimen (unprotected) was held in a Wilmad tissue cell and placed directly into the ESR cavity and subject to 100 s UV-irradiation to establish the background levels of ascorbate radical. The UV source was then blocked and the skin area was marked precisely on the covering silica slide and measured. The sunscreen was applied to the measured area at a range of application levels (quantified by weighing) centred around that recommended in the sunscreen industry (2 mg/cm²). The slide was then placed with the cream-side directly against the skin, again UV-irradiated, and the free radical signal intensity measured. The skin area was restricted to 0.5-1 cm² and the amounts of cream that were applied to the skin were not lower than 0.5 mg to minimise errors due to weighing. This set a lower limit for application of approximately 0.5 mg/cm²; an upper limit of 4 mg/cm² was also chosen. Nine measurements performed in this way were taken for each sunscreen across this range of application.

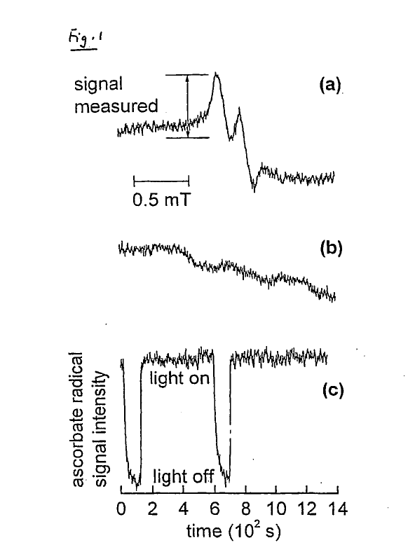

Quantification of the ascorbate radical spectrum was by measurement of the height of the low field absorbance peak relative to the midpoint trough between the doublet signal (marked on FIG. 1(a)). To verify that the signal of any radical species either already present in the sunscreen, or formed as a result of UV-irradiation of the sunscreen alone did not interfere with the signal of the ascorbate radical formed in skin, comparison with the ESR spectra obtained from illumination of sunscreen alone was made (see FIG. 1(b)).

There are two methods that might be used to measure protection: that used, which involves the same skin sample (reducing possible inter-site variation in ascorbate levels), or the comparison of two different pieces of skin both unprotected and protected with cream. The first method not only reduces inter-site variation of ascorbate, but also ensures adequate levels of ascorbate in each skin sample studied (which occasionally could be low in some specimens and believed to be for dietary reasons).

The method requires that ascorbate is sufficiently stable in the skin over the 100 s period between the two measurements. This was verified in fresh skin by studies of the ascorbate depletion rates in skin stored for different periods: skin used immediately after excision (practically within 2-3 hours) showed a very low rate of ascorbate depletion with irradiation; and rates of ascorbate depletion increased with storage time becoming significant at about 3 days refrigerated storage. Whilst every effort was made to use skin immediately after excision, a cut off period of 24 hours was chosen for practical considerations.

To validate the method used, the protection at 2 mg/cm² application was also determined using the second method for one brand of sunscreen. Factor 25 sunscreen (2 mg) was applied to a 1 cm² area to cover five skin samples and the mean radical signal intensity measured at 100 s irradiation (with black tape to shield the back of the skin, see below) was compared to five unprotected skin samples of similar dimensions. The percentage difference in signal intensity was comparable to that determined using the first method (see results below).

It was verified that no significant part of the ascorbate radical signal originated from irradiation scattered through the tissue cell (whose etched lower surface decreases light transmittance considerably compared to that through the transparent cover slip) by irradiating skin protected at the front, and then at the back, by black tape of the same dimensions to the skin sample. The ascorbate radical signal was either abolished or occasionally observed at very low intensities (to about 10% the unprotected signal) when the protective tape was between the incident irradiation and the skin, but when placed behind the skin (to prevent entry into the skin of scattered radiation) the ascorbate radical signal was clearly detected (not shown).

Comparison between two groups of data was undertaken using a Students t-test. All analyses were performed using Sigma Stat™ statistics software, version 2.0 (Jandel Corporation) (available from SPSS Science, 233 S. Wacker Drive, 11^{th} Floor, Chicago, Ill. 60606-6307, Tel: 312-651-3000, www.spssscience.com)

Results

Test Data

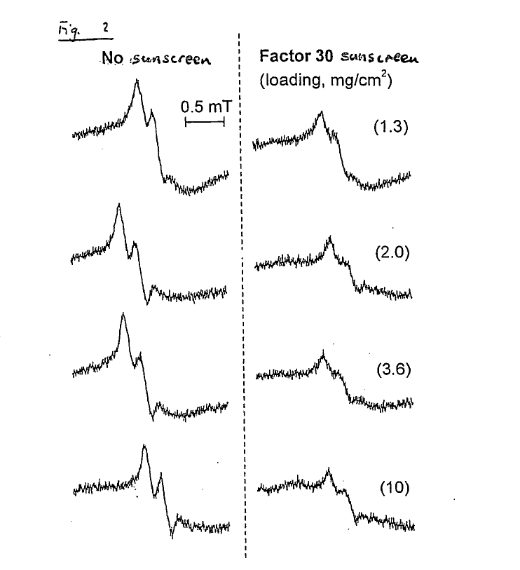

UV-irradiated skin produced the recognisable ESR spectrum (FIG. 1(a) and FIG. 2, left-hand column) of the ascorbate radical (characterised by hyperfine splitting a(H)=0.17 mT [Jurkiewicz, B. A., and G. R. Buettner (1996) EPR detection of free-radicals in UV-irradiated skin: mouse versus human. Photochem. Photobiol. 64, 918-922].

As shown in FIG. 1(b), the radicals formed in sunscreen alone did not interfere with the ascorbate radical at application levels up to about 4 mg/cm²; however, at applications greater than this, radical production in the sunscreen itself becomes appreciable. The ascorbate radical signal decreased very slowly with prolonged irradiation (when studied over a period of 30 min) with the rate of decrease varying between skin samples. Steady state radical production was verified in skin samples used within the twenty-four hour period following excision, as shown in FIG. 1(c). Therefore, any reduction in signal intensity, on the second scan (when sunscreen is applied to the skin) will reflect protection by the sunscreen.

Quantitation of the Protection Provided by High Factor Sunscreens

FIG. 2 shows electron spin resonance spectra obtained, before and after sunscreen (brand 1, factor 30) application to breast skin. These data are typical of that used for subsequent quantitative analysis of the protection provided by high factor sunscreens. There was generally only a small observable reduction in the signal intensity of the ascorbate radical when compared to unprotected skin. This reduction appeared to peak at approximately 2 mg/cm², with little further reduction at greater application levels. Notably, at high application levels the ascorbate radical could still be easily detected.

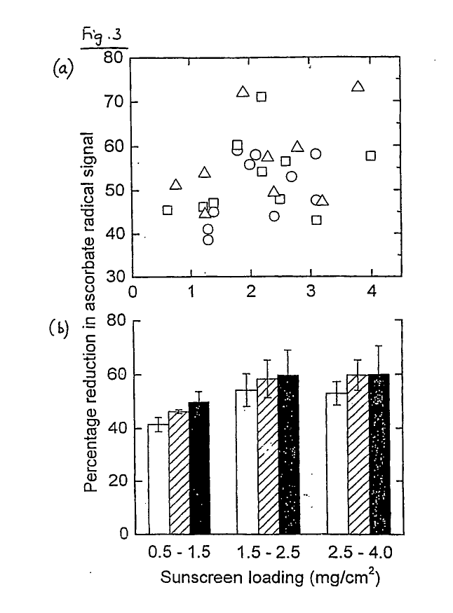

FIG. 3(a) shows a plot of the percentage reduction in signal intensity of the ascorbate radical (taken as the height of the low field absorption relative to the midpoint of the spectrum, FIG. 1(a)) of the ascorbate radical at different applications of high factor sunscreens (all three brands). The percentage reduction in signal intensity is similar for all three brands studied and increases with application up to approximately 2 mg/cm² with no further protection apparent at higher application. Despite use of skin from one anatomical region to cut down on inter-site variation, there is still variability in the protection measured at constant application: although this could result from unavoidable errors associated with the experimental procedure and analysis, it could also result from individual variations in skin architecture that affect the protection provided by the sunscreen.

A rank plot of the data in FIG. 3(a) is shown in FIG. 3(b): the mean percentage reduction in signal intensity of the ascorbate radical was calculated for levels of application 0.5-1.5, 1.5-2.5 and 2.5-4 mg/cm², and the standard deviation calculated for n=3 for each sunscreen at each level of application. It was found that the level of protection at each application is comparable for the three brands studied: the protection at 1.5-2.5 mg/cm² and above is approximately 50-60% reduction in signal intensity; however, at 0.5-1.5 mg/cm² protection is less, between 40-50% for the three brands. Statistical analysis of the data showed that for both brands 1 and 2 the protection at 0.5-1.5 mg/cm² application is significantly different (p=0.034 and 0.024 respectively) from the protection at 1.5-2.5 cm². For brand 3 the protection is not significantly different with application, and brand three appears to provide more protection at lower application than brands 1 and 2 (although this is not statistically significant). Brand 3 had different viscosity to brands 1 and 2 and might explain this. Data at 2.5-4.0 mg/cm² application is not significantly different from that at 1.5-2.5 mg/cm² (for all three brands).

### CONCLUSIONS

It is concluded from these results that Caucasian skin is only protected to about 55% against free radical production by high factor suncreams at the recommended application of 2 mg/cm² at a fluence comparable to relatively weak solar irradiation intensity (equivalent to that measured on a sunny day, June-September, London). At 0.5-1.5 mg/cm² this protection is about 45% reduction in the UV-induced radical concentrations and could be considerably less at lower applications. The irradiation delivered to the skin in these experiments is 1.3 mJ/cm²/s UVA for 100 s. 1 MED is about 20-30 mJ/cm² UVB for a Caucasian: since UVB is 10% sunlight, 1MED UVB will be associated with 225 mJ UVA (which causes 0.001 the erythemal response of UVB). The radiation used in these experiments (1.3 mJ/cm²/s UVA) is equivalent to 130 mJ/cm² UVA (for 100 s irradiation). Thus the UVA radiation dose in our experiments is estimated to be equivalent to sunlight of MED equal to about 0.6, ie. to be sub-erythemal.

The protection currently provided by sunscreens is indicated by the erythema-based SPF measurement, that is, the factor by which the dose for minimal erythema (MED) is increased with protection under standard solar-simulated conditions (or the factor by which sunlight exposure time is increased, before burning, with sunscreen compared to without protection). On the basis of the results in this study it is possible to calculate a UVA sun protection factor (UVASPF) or “free radical protection factor” (FRPF) as the length of UV exposure time with sunscreen protection, compared to without protection, to achieve the same amount of UV penetrating the skin. Since the radical signal intensity is approximately halved at the test level of application (2 mg/cm²), the results suggest that, using this test method, the same UV dose will be achieved in twice the length of duration of exposure, thus suggesting a UVASPF or FRPF of about 2 (and less than 2 at typically used levels of application). Our results are also consistent with previous reports [Azizi, E. et al. (2000) Use of sunscreen is linked with elevated naevi counts in Israeli school children and adolescents. Melanoma Res., 10, 491-8] that, at typical levels of application (measured to be 0.5 mg/cm²) [Bech-Thompsen, N., and H. C. Wulf (1992-93) Sunbathers application of sunscreen is probably inadequate to obtain the sun protection factor assigned to the preparation. Photodermatol Photoimmunol Photomed. 9, 242-4], the protection is less than that measured at the recommended level of application of 2 mg/cm². A free radical protection factor of 2 is substantially less than the erythema-based protection factors for these creams (20-30) and suggests users of these creams will be disproportionately exposed to UVA (which is 90% sunlight).

These results suggest that certain popular high factor sunscreens provide, at most, approximately 60% protection when applied directly to the skin at the recommended amount of 2 mg/cm² and less at lower application (at a fluence comparable to solar UV). The use of differential electron spin resonance to measure directly the UV-induced free radical production in human skin is a rapid and useful method to measure the free radical/UVA protection. Because typical levels of application are generally less than that recommended, the results suggest that sunscreen users are little protected against UVA free radical production. In particular, since the existing sunscreen SPF provides a measure of the protection principally against UVB-induced erythema, users of high factor sunscreens may have an artificial sense of security that they are protected similarly against UVA as to UVB exposure. Thus, as already raised as a concern, use of high SPF factor sunscreens may paradoxically be associated with increased skin cancer risk. There is an urgent need for the validation of UVA SPF measurements of commercial sunscreens, for the UVA protection to be reassessed by sunscreen manufacturers to reflect the protection at typical levels of application, and also for the production of more effective UVA sunscreens at these levels of application. In the absence of adequate UVA protection currently provided by sunscreens, it is recommended that Caucasians (especially those with fairer skill types) avoid prolonged sunbathing, even though “protected” by sunscreens, as the use of sunscreens is likely to increase their risk of UVA-induced carcinogenesis and ageing. Sunscreens do not seem to give adequate protection against these adverse effects of sunlight; in fact they may give a false sense of security to the sunbather that skin is protected when it is not.

The above description broadly describes the present invention without limitation. Variations and modifications as will be readily apparent to those skilled in this art are intended to be included within the scope of this application and subsequent patent(s).

1. A method for measuring the effectiveness of a sunscreen composition or other skin preparation in reducing the exposure of human skin to UVA radiation, the method comprising:
irradiating a sample of human skin or of an effective substitute therefor (herein: “skin”), shielded with the sunscreen composition or other skin preparation to be tested, with UV radiation comprising UVA wavelengths, and determining by electron spin resonance (ESR) spectroscopy the level of induced production of ascorbate radical in the shielded skin; and
determining a quantitative measure of the effectiveness of the sunscreen composition in reducing the exposure of human skin to UVA radiation by comparison of the said level of ascorbate radical production in the shielded skin with the level of ascorbate radical production induced in reference skin under substantially quantitatively comparable conditions.

2. A method according to claim 1, wherein the level of ascorbate radical production induced in reference skin under substantially quantitatively comparable conditions is measured by irradiating a sample of reference skin with UV radiation comparable to that used to irradiate the test sample, in the absence of the sunscreen composition or other skin preparation, and determining by ESR spectroscopy under ESR conditions comparable to those for the test sample the level of induced production of ascorbate radical in the reference skin.

3. A method according to claim 1, wherein the level of ascorbate radical production induced in reference skin under substantially quantitatively comparable conditions is measured by irradiating a sample of reference skin, shielded using a shield having known UVA absorbing characteristics, with UV radiation comparable to that used to irradiate the test sample, and determining by ESR spectroscopy under ESR conditions comparable to those for the test sample the level of induced production of ascorbate radical in the reference skin.

4. A method according to claim 1, wherein the skin sample used for the test and the reference skin sample are the same.

5. A method according to claim 1, wherein the skin sample used for the test is different from the reference skin sample, the two samples being functionally comparable under the ESR conditions used.

6. A modification of a method according to claim 1, wherein in place of, or alongside, the determination of the level of induced production of ascorbate radical in the skin there is determined by ESR spectroscopy the level of UV-induced production of one or more other measurable radical in the skin in the unshielded and shielded skin.

7. A method according to claim 1 or claim 6, wherein the skin sample and the reference skin are irradiated with the UV radiation in the presence of a spin trap molecule capable of forming with the ascorbate and/or with the said one or more other radical an adduct which is substantially quantitatively stable over a lifetime of at least about 100 s and is measureable using ESR spectroscopy.

8. A method according to claim 1 or claim 6, wherein the UV radiation is radiation having a wavelength in the range between about 4 and 400 nm and the UVA radiation is UV radiation in the wavelength range 320-400 nm.

9. Apparatus for testing the effectiveness of a sunscreen composition or other skin preparation in reducing the exposure of human skin to UVA radiation, the apparatus comprising:
at least one sample of human skin or of an effective substitute therefor (herein: “skin”);
a source of UV radiation comprising UVA wavelengths;
means for determining by electron spin resonance (ESR) spectroscopy the level of induced production of ascorbate or other measureable radical in a skin sample on exposure of the skin to the UV radiation;
means for shielding a skin sample with the sunscreen composition or other skin preparation to be tested; and
means for determining a quantitative measure of the effectiveness of the sunscreen composition or other skin preparation in reducing the exposure of human skin to UVA radiation by comparison of the level of ascorbate or other measureable radical production in the shielded skin with the level of ascorbate or other measureable radical production induced in reference skin under substantially quantitatively comparable conditions.

10. An apparatus according to claim 9, wherein the radical is ascorbate.

11. An apparatus according to claim 9, wherein the UV radiation is radiation having a wavelength in the range between about 4 and 400 nm and the UVA radiation is UV radiation in the wavelength range 320-400 nm.

12. A method for assigning a UVA sun protection factor (UVASPF) or free radical protection factor (FRPF) to a sunscreen composition or other skin preparation, which method comprises measuring the effectiveness of the sunscreen composition or other skin preparation in reducing the exposure of human skin to UVA radiation, using the method of any one of claims 1 to 8 or the apparatus of claim 9 or claim 10 or claim 11, expressing the said effectiveness as the fraction (f) of unshielded UVA-induced ascorbate radical production exhibited by the shielded skin, and assigning the UVASPF or FRPF to the composition or preparation by virtue of the relationship:
UVASPF or FRPF=1/f.

13. A sunscreen composition or other skin preparation comprising a UVASPF or FRPF that has been assigned according to the method of claim 12.

14. A sunscreen composition or other skin preparation according to claim 13, preferably for application to the skin at least once per day, wherein the assigned UVASPF or FRPF is above the safe minimum UVASPF or FRPF for the latitude, season and/or climate in which the composition or preparation is to be used, calculated having regard to a safe maximum daily exposure to UVA radiation and an assumed, expected or likely actual daily exposure to UVA radiation at that latitude, season and/or climate.

15. A method for measuring the effectiveness of a sunscreen composition or other skin preparation in reducing the exposure of human skin to UVA radiation comprising the step of using differential ESR spectroscopy to determine an induced ascorbate radical production level.

16. The method according to claim 15, wherein the differential ESR spectroscopy is used to quantify UVA-induced ascorbate radical production in skin shielded by the said composition or other skin preparation, in comparison with reference, preferably unshielded, skin.

17. The method according to claim 15, wherein the UVA radiation is UV radiation in the wavelength range 320-400 nm.
